(19) United States
(12) Patent Application Publication
(10) Pub. No.: US 2011/0288199 A1
(43) Pub. Date: Nov. 24, 2011

(54) Fiber-Hydrogel Composite for Tissue Replacement

(75) Inventors: Anthony M. Lowman, Wallingford, PA (US); Giuseppe R. Palmese, Hainesport, NJ (US); Suzanne A. Maher, New York, NY (US); Russell F. Warren, Greenwich, CT (US); Timothy M. Wright, New York, NY (US); Julianne L. Holloway, Philadelphia, PA (US)

(73) Assignees: Hospital for Special Surgery; Drexel University

(21) Appl. No.: 12/783,393

(22) Filed: May 19, 2010

(51) Int. Cl. A61L 27/48; A61F 2/44; A61F 2/38; A61F 2/08; A61F 2/02; A61F 2/28

(52) U.S. Cl. 523/114; 523/113; 523/115

(57) ABSTRACT

The present invention includes tailored fiber-reinforced hydrogel composites for implantation into a subject. The present invention also includes systems and methods for controlling the relative percent volume of the hydrogel and fibers, cross-linking, fiber orientation, weave and density, such that the material properties of the composite can be controlled and/or customized to match particular tissue types. The composites of the present invention are suitable for repairing or replacing musculoskeletal tissues and/or fibrocartilage, such as the meniscus, ligaments and tendons.

### STATEMENT REGARDING FEDERALLY SPONSORED RESEARCH OR DEVELOPMENT

This invention was made with support from the U.S. Department of Defense through the National Defense Science and Engineering Graduate Fellowship, in addition to the U.S. Army Research Laboratory through the Army Materials Center of Excellence Program, contract W911NF-06-2-0013. The government has certain rights in the invention.

### BACKGROUND OF THE INVENTION

The menisci are C-shaped fibrocartilage disks with a cross-sectional wedge-shape that occupy the periphery of the knee joint, as depicted in prior art FIG. 1. The anterior and posterior horns of the menisci are connected to the tibial plateau by ligaments that insert into the intercondylar regions. During loading, radial (extrusive) forces are resisted by these firm attachments, particularly to the tibia at the anterior and posterior horns. This produces large circumferentially oriented hoop tensile stresses within the meniscus.

Sometimes the meniscus cannot withstand the mechanical burdens placed upon it, and it tears. Traumatic tears are most commonly observed in young, athletically active individuals (Rodkey, 2000, Instr Course Lect 49:189-93), while degenerative meniscal tears tend to occur in patients over the age of 40. In cases where meniscal preservation or repair is impossible, a meniscectomy (partial or total surgical removal of a meniscus) is performed. Unfortunately, the deleterious effects of meniscectomy include limited mobility, pain and ultimately osteoarthritic degradation (Allen, et al., 1984, J Bone Joint Surg Br 66-B:666-671; Fairbank, 1948, J Bone Joint Surg Am. 30B:664-670; Johnson, et al., 1974, Journal of Bone and Joint Surgery—American 56A: 719-729), and the subsequent deterioration of the joint leads to disability, the need for multiple surgeries, and oftentimes total knee joint replacement. Despite these risks, approximately 850,000 surgical procedures of the meniscus are performed each year in the United States alone (Rodkey, 2000, Instr Course Lect 49:189-93), and as the current population ages this number will likely rise. With such a prevalence of meniscal degeneration, a meniscectomy that leads directly to late-stage osteoarthritis and total knee replacement is an undesirable treatment option. However, providing a meniscal substitution could delay or even avoid the onset of osteoarthritis.

Current options for meniscal substitution include allografts, autografts, bioresorbable scaffolds and synthetic prostheses. Unfortunately, each of these options has significant drawbacks. For example, in the case of allografts, a cryopreserved sterilized meniscus from a donor is transplanted into the patient. This is a common procedure in appropriately selected patients, such as those with reasonable cartilage preservation and symptomatic meniscal deficiency. However, problems with allografts include transmission of infectious diseases and the difficulty of matching the donor meniscal shape to that of the recipient. Autografts are tendon/ligament/fat pad grafts that are transplanted from a remote location into the knee joint, but these have produced disappointing results in the mid- to long-term (Goble, et al., 1999, Scand J Med Sci Sports. 9(3):168-76; Johnson, et al., 2000, Arthroscopy. 16(2):191-6). Bioresorbable scaffolds are designed to encourage tissue ingress while gradually resorbing, but the ability of bioresorbable scaffolds to carry joint contact loads is questionable, and there is little mechanical test data to support their use, as few have been tested in the joints of large weight bearing animals. A variety of synthetic prostheses have been suggested as meniscal substitutes. For example, Teflon meniscal implants have been used in dogs (Toyonaga et al., 1983, Clin Orthop Relat Res. 179: 291-7.), polyester-carbon fiber prostheses have been used in rabbits (Wood, et al., 1990, Biomaterials 11:13-16.), and a combination of Dacron and Teflon with a polyurethane coating have been used in rabbits (Messner, et al., 1993, J Biomed Mater Res 27: 1165-73 and Messner, 1994, Biomaterials. 15: 223-30). Problems encountered for such implants include fibrillation (Wood, et al., 1990, Biomaterials 11:13-16), cartilage softening, formation of osteophytes (Messner, et al., 1993, J Biomed Mater Res 27: 1165-73), and uncontrolled adhesion between the implant and the popilteal muscle (Toyonaga et al., 1983, Clin Orthop Relat Res. 179: 291-7). Kobayashi (Kobayashi, et al., 2005, Biomaterials, 26:3243-3248) tried to mimic meniscal compressive properties by modifying the water content of a poly(vinyl alcohol) hydrogel and exhibited satisfactory chondroprotection when compared to the meniscectomized knee in 2 year pilot dog model. However, the tensile properties of hydrogels are poor, and the long-term performance of such a replacement, especially in light of the high tensile stresses that menisci must endure, is questionable.

However, hydrogels do possess compressive properties that have the potential of matching many native soft tissues, such as the nucleus pulposus (Joshi, et al., 2005, Biomaterials 27:176-18, Gomes, et al., 2004, Journal of Applied Biomaterials. 69:135-140, Thomas, et al., 2003, J. Biomed. Mater. Res., 67:1329-1337). Such features have lead scientists to propose the use of hydrogels in cartilage repair (Peppas, et al., 1977, J. Biomed. Mater. Res., 11; 423-434; Stammen, et al., 2001, Biomaterials 22, 799-806) and meniscal replacements (Kobayashi, et al., 2005, Biomaterials, 26:3243-3248). In particular, poly(vinyl alcohol) (PVA) based hydrogels have been considered ideal for biomaterial applications, and have been investigated extensively for articular cartilage applications [Bray et al., 1973, J Appl Polym Sci. 17:3779-94; Katta et al., 2007, J Biomed Mater Res A. 83(2):471-9; Maher et al., 2007, J Biomed Mater Res A. 83(1):145-55; Grant et al., 2006, Biotechnol Prog. 22(5):1400-6; Spiller et al., 2009, J Biomed Mater Res B. 20(2):752-9]. However, poor properties in tension continue to limit the actual use of this material in the treatment and/or repair of fibrous tissues, because the tensile properties of hydrogels are poor and far inferior to that of musculoskeletal tissues, such as the meniscus of the knee. Therefore, these proposals have not been met with great success.

Thus, meniscal allografts are plagued by the threat of transmission of infectious diseases, the difficulty of matching meniscal shape, and continued problems with maintaining cell viability. Autografts cannot match either meniscal shape or material properties, and degradable constructs either have poor mechanical properties or require that significant areas of the meniscus remain intact. Although the concept of using synthetic non-degradable materials has been explored in animal models, no material has as yet been proposed that is capable of being tailored to match both the compressive and tensile properties of the native meniscus, or for that matter, any musculoskeletal tissue. There are significant challenges to mimicking the complex mechanical properties of native soft tissues in a synthetic substitute, and therefore, there is no clinically available implant to replace damaged musculoskeletal soft tissues such that their pre-injury function is restored. This absence negatively impacts the treatment of patients with debilitating orthopedic injuries and in many instances leads to post-traumatic arthritis in the affected joint. The present invention addresses these needs by providing a composite whose mechanical properties can be readily controlled to match that of the native tissue, thereby increasing the likelihood that the implant can function much in the way of the native tissue.

### SUMMARY OF THE INVENTION

The present invention relates to a fiber-reinforced hydrogel composite that mimics a native tissue of a mammal and is suitable for implantation in the mammal. The composite includes at least one fibrous component forming part of a fiber volume fraction, and a polymer fraction comprising poly(vinyl alcohol) (PVA) and poly(acrylic acid) (PAA), wherein the ratio of PVA to PAA is altered to control the mechanical properties of the resulting composite. In one embodiment, the mechanical properties of the resulting composite are controlled by altering the amount of cross-linking agent used in cross-linking components of the composite.

The present invention also relates to a device for implantation within a subject that includes at least one fibrous component forming part of a fiber volume fraction and at least one hydrogel component forming part of a polymer fraction, wherein the device is subjected to a plurality of freeze-thaw cycles, such that the mechanical properties of the device are controlled based upon the number of freeze-thaw cycles to which the device is subjected. In another embodiment, the mechanical properties are further controlled based upon the duration and rate of freezing and thawing during the freeze-thaw cycles.

In another embodiment, at least one fibrous component comprises an ultra high molecular weight polyethylene (UHMWPE). In another embodiment, at least one fibrous component comprises polypropylene (PP). In another embodiment, the fiber volume fraction in the composite is within about 5-50%, and the polymer fraction in the composite is within about 20-50 wt %. In another embodiment, at least one of the diameter, orientation, number and volume fraction of at least one fibrous component is altered to impart directional, anisotropic and inhomogeneous modulus to the composite to produce a tensile modulus range of about 0.25 MPa to 250 MPa, wherein the fiber volume fraction ranges from between about 0% to 28%. In another embodiment, the composite osmolality is balanced via the polymer fraction to equilibrate with the mimicked native tissue. In another embodiment, at least one fibrous component is used for attachment upon implantation within the mammal. In another embodiment, the composite replaces a musculoskeletal tissue of the mammal. In another embodiment, the composite augments a damaged or degraded musculoskeletal tissue of the mammal. In another embodiment, the composite replaces the meniscus of the mammal. In another embodiment, the composite replaces a ligament or a tendon of the mammal. In another embodiment, the composite is used for closure of the annulus pulposus. In another embodiment, the composite further comprises a porous periphery to augment attachment within the mammal via an ingrowth of surrounding tissue.

The present invention also relates to a fiber-reinforced hydrogel composite that mimics a Native tissue of a mammal and is suitable for implantation in the mammal, including at least one fibrous component forming part of a fiber volume fraction and at least one hydrogel component forming part of a polymer fraction, wherein the mechanical properties of the composite is controlled based on the selection of at least one hydrogel component. In another embodiment, the selection of at least one hydrogel component is based on hydrogel viscosity. In another embodiment, the selection of at least one hydrogel component is based on hydrogel stability, such that the hydrogel does not substantially swell or shrink when implanted within the mammal. In another embodiment, the selection of at least one hydrogel component is based on its reaction with the surface of at least one fibrous component. In another embodiment, the mechanical properties of the composite is further controlled based on the selection of at least one fibrous component. In another embodiment, the selection of at least one fibrous component is based on the ability to weave at least one fibrous component in a controlled fiber orientation. In another embodiment, the selection of at least one fibrous component is based on the ability to modify the surface of at least one fibrous component, such that it interacts with at least one hydrogel component.

The present invention also relates to a fiber-reinforced hydrogel composite that mimics a native tissue of a mammal and is suitable for implantation in the mammal, including at least one fibrous component forming part of a fiber volume fraction and at least one hydrogel component forming part of a polymer fraction, wherein the hydrophobic and hydrophilic interactions at the interface of at least one fibrous component and at least one hydrogel component are maximized, and the in vivo swelling of the composite is minimized.

The present invention also relates to a method of controlling the mechanical properties of a fiber-reinforced hydrogel composite that mimics a native tissue of a mammal and is suitable for implantation in the mammal. The method includes the steps of selecting at least one fibrous component in a fiber volume fraction and at least one hydrogel component in a polymer fraction, such that the hydrophobic and hydrophilic interactions at the interface of at least one fibrous component and at least one hydrogel component are maximized and in vivo swelling of the composite is minimized.

The present invention also relates to a method of controlling the mechanical properties of a fiber-reinforced hydrogel composite that mimics a native tissue of a mammal and is suitable for implantation in the mammal, including the steps of adding at least one fibrous component in a fiber volume fraction and adding a plurality of hydrogel components in a polymer fraction, wherein the plurality of hydrogel components includes poly(vinyl alcohol) (PVA) and poly(acrylic acid) (PAA), and altering the ratio of PVA to PAA to control the mechanical properties of the resulting composite.

The present invention also relates to a method of controlling the mechanical properties of a fiber-reinforced hydrogel composite that mimics a native tissue of a mammal and is suitable for implantation in the mammal, including the steps of adding at least one fibrous component in a fiber volume fraction and adding at least one hydrogel component in a polymer fraction, subjecting the composite to a plurality of freeze-thaw cycles, such that the mechanical properties of the composite are controlled based upon the number of freeze-thaw cycles the composite is subjected to.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a prior art depiction of a front view and superior view of the medial and lateral menisci.

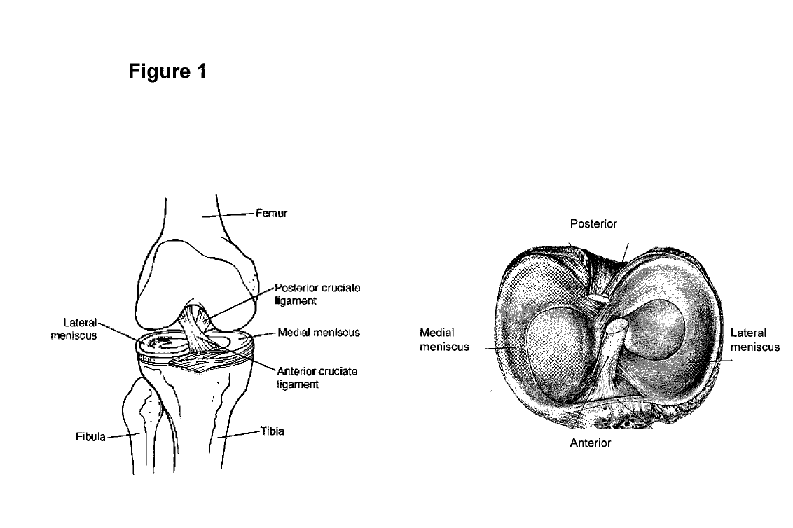

FIG. 2 depicts the compressive modulus of 10, 20, 30, and 35 wt % PVA hydrogels with increasing freeze-thaw cycles. No significant changes were observed after the first six cycles (p<0.05). Trend lines ( - - - ) represent a linear best fit through the first six cycles following by the average value within the plateau region. Hydrogels with 20, 30, and 35 wt % PVA can be modified to approximate the same stiffness as the human meniscus.

FIG. 3 depicts the tensile modulus of 10, 20, 30, and 35 wt % PVA hydrogels with increasing freeze-thaw cycles. Increases in modulus were observed through the first six cycles. Trend lines ( - - - ) represent a linear best fit through the first six cycles following by the average value within the plateau region.

FIG. 4 depicts the Mc from swelling studies using the Bray and Merrill equation for 10, 20, 30, and 35 wt % PVA hydrogels, showing a decreasing trend with freeze-thaw cycles and indicating increases in cross-link density.

FIG. 5 depicts bound water as a function of freeze-thaw cycles as determined using DSC (differential scanning calorimetry). Decreases in the amount of bound water present in PVA hydrogels with cycling indicates hydrogen bonding occurring between PVA chains.

FIG. 6 depicts micrographs of 20 wt % PVA hydrogels in the hydrated state, showing structural remodeling of PVA hydrogels occurs with increasing freeze-thaw cycling in the form of polymer densification. Scale bars indicate 80 μm.

FIG. 7 depicts the tensile modulus of PP-PVA composites increasing linearly with fiber volume fraction.

FIG. 8 depicts the tensile modulus of UHMWPE-PVA composites increasing linearly with fiber volume fraction within the range of native meniscus.

FIG. 9 depicts micrographs of 20 wt % PVA hydrogels showing polymer rich zones occurring near UHMWPE interface with increasing freeze-thaw cycling. Scale bars indicate 80 μm.

FIG. 10 depicts a resin transfer molding process.

FIG. 11, comprising FIGS. 11a and 11b, depict an interface created during a multi-stage resin transfer molding process between hydrogel created in the first stage verses second stage.

FIG. 12, comprising FIGS. 12a and 12b depict images of a fiber-hydrogel composite. FIG. 12a depicts a SEM of PVA-Kevlar composite, in which a cross hatch pattern of Kevlar fibers embedded within the hydrogel matrix is seen. FIG. 12b depicts a magnified image of individual fibers illustrating fiber diameters ranging from 5-10 microns.

FIG. 13, comprising FIGS. 13a and 13b, depict aspects of surface modification. FIG. 13a depicts surface modification using plasma treatment, while FIG. 13b depicts equipment for surface modification.

FIG. 14 depicts the gross appearance of plasma treated and untreated samples.

FIG. 15 depicts in vitro swelling of PVA hydrogels formed after five freeze-thaw cycles. The swelling ratio was calculated as initial hydrogel mass to swollen hydrogel mass. The degree of deswelling is shown to decrease with increasing polymer concentration.

FIG. 16 depicts in vitro swelling of PVA hydrogels formed after 2 and 4 freeze-thaw cycles at concentrations in between 20 and 40 wt % PVA. Limited swelling is observed in between 30 and 35 wt % PVA.

FIG. 17 depicts XPS results showing increased formation of oxygen containing groups with increasing oxygen plasma treatment time. Oxygen plasma treatments were performed at a flow rate of 1 LPM (liters per minute) on UHMWPE fibers.

FIG. 18 depicts fiber pull-out tests performed on 20 wt % PVA hydrogels synthesized using five freeze-thaw cycles. Results indicated an increase in interfacial shear strength with increasing oxygen plasma treatment time.

FIG. 19 depicts SEM micrographs showing increasing pitting and roughening of the fiber surface with increased oxygen plasma treatment time. Oxygen plasma treatments were performed on UHMWPE fibers at 1 LPM for 0 (a), 1.5 (b), and 2 (c) minutes.

FIG. 20 depicts roughness measurements of the UHMWPE fiber surfaces performed using AFM. Oxygen plasma treatments were operated at a flow rate of 1 LPM and up to eight minutes. As shown, increasing roughness was also noted with increasing time, as seen with SEM.

FIG. 21 depicts fiber pull-out tests performed on 20 wt % PVA hydrogels formed after five freeze-thaw cycles indicate an increase in pull out frictional force following plasma treatment. This is representative of a roughening of the fiber surface from treatment.

FIG. 22 depicts PVA-UHMWPE composites tested at volume fractions up to 28% UHMWPE showing increases in tensile modulus of 0.25 MPa (0% UHMWPE) to 250 MPa.

### DETAILED DESCRIPTION

The present invention combines a fiber composite technology with that of hydrogel chemistry to control the material properties of the resulting implant over a wide spectrum, allowing for the replacement of a damaged meniscus, ligament, tendon or other native tissue, with a construct that functionally mimics the native tissue. The composites of the present invention include at least one fibrous component forming part of a fiber volume fraction, and a polymer fraction comprising poly(vinyl alcohol) (PVA) and poly(acrylic acid) (PAA), wherein the ratio of PVA to PAA is altered to control the mechanical properties of the resulting composite. The mechanical properties of the resulting composite can be controlled by altering the amount of cross-linking agent used in cross-linking components of the composite.

The present invention also relates to a device for implantation that includes at least one fibrous component and at least one hydrogel component, wherein the device is subjected to a plurality of freeze-thaw cycles, such that the mechanical properties of the device are controlled based upon the number of freeze-thaw cycles to which the device is subjected. The mechanical properties can be further controlled by altering the duration and rate of freezing and thawing during the freeze-thaw cycles.

The present invention also provides systems and methods of uniquely tailoring the properties of the composite to match that of a broad spectrum of tissues, particularly musculoskeletal tissues.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “hydrogel” as used herein refers to a network of oligomers or polymer chains that are water-insoluble, sometimes found as a colloidal gel in which water is the dispersion medium. Hydrogels are natural or synthetic polymers that show superabsorbent properties (having even over 99% water).

The term “polymer” as used herein refers to a large molecule (macromolecule) composed of repeating structural units often connected by covalent chemical bonds. Polymers may be natural or synthetic. Polymer nomenclature is generally based upon the type of monomer residues comprising the polymer. Polymers that contain only a single type of repeat unit are known as homopolymers, while polymers containing a mixture of repeat units are known as copolymers.

The term “fiber” as used herein refers to a class of materials that are continuous filaments or are in discrete elongated pieces. They can be used as a component of composite materials, or matted into sheets. They can further be used in the manufacturing of composites via cross-linking, gluing, weaving, braiding, knitting, knotting, molding and the like. The term “composite” as used herein means a construct of either hydrogel components or a construct including both a fiber component and a hydrogel component. The composites are designed for use in the repair and/or replacement of various tissue types.

The term “subject” as used herein refers to any living animal, preferably a mammal and more preferably a human. The composites as described herein are designed for implantation within a subject in need thereof.

### Description

The present invention provides a fiber-hydrogel composite for the replacement or repair of musculoskeletal tissues. The addition of fibers reinforces the tensile properties of the material, while the hydrogel matrix maintains suitable compressive properties. The present invention also provides systems and methods for controlling the relative percent volume of the hydrogel and fibers, fiber orientation, weave and density, such that the material properties of the composite can be controlled and/or customized to match particular tissue types.

### Hydrogels

Hydrogels can be classified into physical and chemical cross-linked gels. Both of these types of gels can be modified for use in a composite based on the selection criteria discussed herein. In certain embodiments, interpenetrating networks can also be used to combine properties from multiple hydrogel systems. In other embodiments, the resulting composite osmolality can be balanced via the hydrogel component to equilibrate with the native tissue.

As contemplated herein, various hydrogels consisting of copolymers based on PVA and Poly(acrylic acid) (PAA), serve as examples of chemically cross-linked hydrogels suitable for use in the present invention. PAA is an anionic polymer that can be synthesized into hydrogel networks via radical polymerization of the vinyl, AA monomer with a multifunctional cross-linking agent. PAA has also been used in soft tissue repair (Stiles, et al., 2002, Biomacromolecules, 3:591-600) as well as in bone tissue repair (Abusafieh, et al., 1997, J Biomed Mater Res. 38(4):314-327). As contemplated herein, the charge density and swelling properties can be widely varied in a controlled and/or selective manner, according to methods and systems of the present invention. The addition of PAA to PVA can provide a resultant material with a negative charge, which is similar to natural tissue, such that it will prevent deswelling due to osmotic variations of the surrounding tissues, while the PVA can provide for elastic reinforcement. In certain embodiments, the polymer weight fraction in the hydrogel is within about 20-50%.

Other examples of hydrogel material for use with the present invention may include, without limitation, hydrogels based on segmented polyurethanes or polyureas, poly(methacrylic acid) and poly(hydroxyethyl methacrylate), polyacrylamides, polyethylene oxide and polyvinyl pyrrolidones (PVP), and any combination of such hydrogel components. In certain embodiments, the hydrogels may be non-biodegradable and/or porous, and in further embodiments, where the composite can be augmented with a porous periphery, or open-cell pores, to augment attachment via the ingrowth of surrounding tissue, if desired.

### Fibers

To enhance and add desirable properties to the hydrogel, a variety of polymer fibers, including ultra high molecular weight polyethylene (UHMWPE) and polypropylene (PP) fibers can be used to reinforce the hydrogel matrix. In one embodiment, Kevlar™ (DuPont) and/or Spectra™ (Honeywell) fibers may be used. Both Kevlar and Spectra fibers characteristically have high strength, stiffness, toughness and good thermal stability. For example, Spectra fibers possess high strength (˜3 GPa), very high tensile modulus (range of about 97 to 113 GPA) and an elongation at break of up to approximately 3.5%. It should be appreciated that other types of fiber, as would be understood by those skilled in the art, may be used in the present invention. All suitable fibers may possess similar tensile strength, such as around 2.3 GPa, but each may have significantly different tensile modulus and elongation at break, providing for property variations depending on the class of fiber selected.

It should also be recognized that two or more different types of fibers may be used in combination to further control the final mechanical characteristics of the resulting composite. For example, where the fibers are used for attachment upon implantation, a thicker fiber might be used around the periphery of the implant, to give maximal strength to the periphery and to any anchoring devices. As another example, fibers with harder surfaces might be used in areas of the implant that do not pass through the hydrogel component, while other types of fibers with softer or swellable surfaces might be used for passing through hydrogel portions. Further, a substantially nonstretching fiber might be used to give a high level of strength along an axis, arc, or surface of an implant, while a different type of elastic and/or stretchable fiber might be used to provide flexibility and elastic resilience along a different axis, arc, or surface. In yet another example, a nonresorbable fiber (made of high-strength polymers) and/or a degradable, resorbable fiber (such as collagen or various known polymers) can be used, either separately or in combination.

In certain embodiments, the fiber volume fraction in the resulting composite can be within about 5-50%. Of course, the final amount of fiber used will depend primarily on whether the fiber is used as an organized or unorganized structure, as well as the final tensile strength desired for the tissue being mimicked.

In another aspect of the present invention, the fibers may be integrated into the composite in a variety of ways, including a random or unorganized integration, or a patterned or organized integration, such as a three-dimensional mesh. In addition to the methods of manufacturing the composites as described herein, other methods may used as would be understood by those skilled in the art, such as crosslinking, gluing, weaving, braiding, knitting, knotting, and molding. The desired shape and customized mechanical properties of the composite will dictate the type or combination of manufacturing techniques used.

### Binding Fiber and Hydrogel

According to an aspect of the present invention, the bond between fibers and hydrogel can be created in a variety of ways. For example, in one embodiment, bonds are created by maximizing the hydrophobic and hydrophilic interactions at the material interface. In another embodiment, bonds are created by maximizing the interaction between reactive groups in the precursor hydrogel mixture and reactive groups on the fiber surfaces physically. In yet another embodiment, bonds are created via cross-reaction between reactive groups in the precursor mixture and on the fiber surfaces to create a cohesive bond between the fiber and hydrogel. Of course, any combinations of such methods are contemplated herein, as well as other bonding techniques available to those skilled in the art. Further, organic fibers, for example, can be readily modified via plasma surface treatments to include high concentrations of chemically active species on the fiber surfaces. Therefore, by introducing reactive moieties into the hydrogel precursor, a stable bond can be created between the fiber and hydrogel.

The mechanical behavior of a fiber-reinforced polymer matrix composite may depend not only on the properties of the two constituents, but also on the strength of the interface between them. The interface governs stress transfer between components that have distinct mechanical characteristics. For example, a weak interface can lead to fiber-matrix debonding upon cyclic load, which can be accelerated by exposure to environmental agents (Drzal, L. T. 1986. “The Interphase in epoxy composites,” Epoxy Resins and Composites II. Berlin and New York, Springer-Verlag:1-32; Karbhari, et al., 1997, Journal of Materials Science. 32(21):5761-5777, Wagner, et al., 1993, Journal of Materials Science, 28(8):2238-2244). To avoid this in the present invention, the fiber-hydrogel interface can be augmented via cross-reactions between the two materials, which may be initiated with plasma treatment or chemical grafting, or via physical interactions between the hydrogel side groups and the fiber surface.

In one embodiment, the interface is enhanced with cross-reactions between reactive groups in the precursor mixture and on the fiber surfaces, where the cross-reaction can be initiated with plasma treatment, the duration of which can be between about 30s and 10 min, and the plasma flow rate can be between about 1 and 10 LPM oxygen gas. In another embodiment, the interface is enhanced with cross-reactions between reactive groups in the precursor mixture and on the fiber surfaces, where the cross-reaction is enhanced with chemical grafting following oxygen plasma treatment. In yet another embodiment, the interface is formed via physical interactions between the hydrogel side groups and the fiber surface, where the physical interaction takes the form of roughened fiber surfaces.

### Methods of Controlling Implant Mechanical Properties

As contemplated herein, the present invention includes systems and methods for controlling the relative percent volume of the hydrogel and fibers, cross-linking, fiber orientation, weave and density, such that the material properties of the composite can be controlled and/or customized to match particular tissue types. The present invention also includes systems and methods for controlling the properties of the end product by providing a tailored structure or geometry to the implant.

For example, in one embodiment, the method for controlling the resulting mechanical properties of the implant is based on altering the ratio of PVA to acrylic acid monomer and/or the amount of cross-linking agent to control the swelling properties due to osmotic changes in the tissues. Without limitation, exemplary components of a chemically cross-linked hydrogel system are identified as illustrated in Table 1.

TABLE 1
Hydrogel components for chemically cross-linked systems

| | | |
|---|---|---|
| Monomer 1 | Hydrophilic, | Use of an anionic, difunctional acrylate |
| | charged molecule | or methacrylate. Two examples include |
| | provides net | Acrylic acid (AA) and methacrylic acid |
| | negative charge | (MAA). Methacrylic acid is more |
| | matching natural | hydrophobic and can provide |
| | tissue | for increased stiffness. |
| Monomer 2 | Neutral molecule | Use of a neutral difunctional acrylate |
| | providing | or methacrylate. Examples include |
| | structural stability | Hydroxyethyl methacrylate (HEMA) |
| | | and methyl methacrylate (MMA). |
| | | MMA is hydrophobic and can provide |
| | | for a significantly stiffer material. |
| Cross- | Provides for | Poly(ethylene glycol) dimethacrylate. |
| linking | permanent | The concentrations of this molecule |
| agent | network | (typically used in the range of about |
| | | 0.1 to 5 mole %) can be varied to |
| | | change the swelling and mechanical |
| | | properties of the network. |
| Solvent | Generation of | Aqueous solutions of methanol/water |
| content in | homogeneous, | can be used as a solvent. Increased |
| the reaction | bubble free | solvent content can lead to softer, |
| | matrices | more swellable networks. |
| Polymer | Viscosity enhancer | Poly(vinyl alcohol) can be useful in |
| | tackifying agent | cartilage and nucleus replacement |
| | | applications due to its elastic |
| | | properties and biocompatibility. |
| | | The addition of the polymer to |
| | | the precursor solutions can provide |
| | | beneficial properties as well as serve |
| | | as a tackifying agent for a molding |
| | | process. Gels can be prepared |
| | | with and without PVA. |

Physically cross-linked PVA hydrogels have been researched extensively for use as biomaterials, including as a replacement for articular cartilage (Bray, et al., 1973, Journal of Applied Polymer Science. 17: 3779-3794; Katta Et Al., 2007, Journal Of Biomedical Materials Research A. 83:471-479; Maher, et al., 2007, Journal of Biomedical Materials Research A. 83:145-155; Grant, et al., 2006, Biotechnol Prog. 22:1400-1406). PVA hydrogels offer the advantage of being physically cross-linked using freeze-thaw cycles, without the need for utilization of potentially toxic cross-linking agents. During freezing, water freezes and causes regions of high concentration of PVA to form. As the PVA chains come in close contact with each other, crystallite formation and hydrogen bonding can occur between the chains. These interactions remain intact following thawing, and thereby create a three-dimensional hydrogel network. Thus, in another embodiment, the method for controlling the resulting mechanical properties of the implant is based on increasing the number of freeze-thaw cycles, such that the amount of hydrogen bonding and crystallite formation can be increased. As a result, the mechanical properties of the hydrogel can be tailored or customized, based on the number of freeze-thaw cycles (Hassan, et al., 2000, Advances in Polymer Science. 153: 37-66; Lozinsky, et al., 2008, Colloid Journal. 70(2): 189-198; Ricciardi, et al., 2004, Macromolecules, 37: 9510-9516; Hatakeyema, et al., 2005, Thermochimica Acta. 431:144-148) used in the process. In a further embodiment, the method for controlling the resulting mechanical properties of the implant is based on controlling the duration and rate of freezing and thawing during the manufacturing process.

In another embodiment, the method for controlling the resulting mechanical properties of the implant is based on selective criteria for choosing suitable hydrogels. For example, in one embodiment, the selection of hydrogel may be based on viscosity, which can be tailored for ease of manufacture. In another embodiment, the selection may be based on stability, such that the hydrogel does not substantially swell or shrink when exposed to a particular physiological environment, or following a molding process to prevent delamination from fibers. In yet another embodiment, the fiber and hydrogel materials are chosen to maximize the hydrophobic and hydrophilic interactions at the interface and to minimize in vivo swelling. In another embodiment, the selection of hydrogel may be based on tensile properties that can be controlled. In yet another embodiment, the selection may be based on its reaction with the surface of the fibers, or a favorable interaction to ensure a chemical or physical attachment between the hydrogel and fibers. It should be appreciated that the present invention is not limited to any particular criteria, and may further incorporate other such criteria or combinations of criteria, as would be understood by those skilled in the art.

In another embodiment, the method for controlling the resulting mechanical properties of the implant is based on selective criteria for choosing suitable fibers. For example, in one embodiment, the selection of fiber may be based on the ability to weave and/or knit the fibers in 2D and 3D patterns or structures. In another embodiment, the selection of fiber may be based on the ability to create mats with random or controlled fiber orientation. In another embodiment, the selection of fiber may be based on the ability to modify the surface of the fibers such that they can interact with the hydrogel in which they are embedded. In yet another embodiment, the selection of fiber may be based on the ability to use the fibers to affix the implant. In yet another embodiment, the fiber diameter and orientation, fiber number and volume fraction can be altered to impart directional, anisotropic and inhomogeneous modulus to the construct to produce a desired tensile strength, such as a tensile modulus range of about 0.25 MPa to 250 MPa, for example, by changing the fiber volume fraction from 0% to about 28%. In yet other embodiments, the method for controlling the resulting properties of the implant is based on altering the geometry or structural features of the implant.

### Tissue Repair and Replacement

As explained hereinthroughout, the fiber-reinforced hydrogel composites may be used for replacing or repairing various musculoskeletal tissues and fibrocartilage, preferably in a mammal, and more preferably in a human. For example, in certain embodiments, the composite can be used for the purposes of augmenting damaged or degraded musculoskeletal tissues. In another embodiment, the composite can be used for replacement of the meniscus. In other embodiments, the composite is used for the replacement of ligaments and tendons, or even muscle immediately connected to tendon. In yet another embodiment, the composite is used for closure of the annulus pulposus.

The fiber-reinforced hydrogel composites may also be used for replacing or repairing other items, such as a secondary implant repairing or replacing a first implant. In other embodiments, the composites of the present invention can be constructed to match an allograft, or any other replacement device as would be understood by those skilled in the art.

It should be appreciated that the composites may be implanted into a patient using operative techniques and procedures understood by those skilled in the art. In certain embodiments, magnetic resonance imaging (MRI) can be used to define and/or model the geometry of the end product for implantation, thereby generating a structure and geometry that is tailored to a particular subject. Further, the operative technique used to prepare the site for implantation can be based on computer navigation and/or computer guided technology.

The methods described herein are by no means all-inclusive, and further methods to suit the specific applications as contemplated herein will be apparent to the ordinary skilled artisan.

### EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in the experiments and examples disclosed herein are now described.

### PVA Hydrogel Synthesis

PVA (99+% hydrolyzed), with a molecular weight of about 89,000-98,000 g/mol, and poly(vinyl pyrrolidone) (PVP), with a molecular weight of about 40,000 g/mol, were obtained from Sigma Aldrich. PVP was added in small amounts to the hydrogel formulation to improve network stability through interchain hydrogen bonding [Thomas et al., 2003, J Biomed Mater Res A. 67(4):1329-37]. Polymer solutions were prepared by mixing about 10 to 20 wt % polymer, composed of approximately 99 wt % PVA and 1 wt % PVP, in deionized water. The container was sealed and autoclaved at about 121° C. for two hours to ensure complete dissolution of PVA. Polymer solutions were poured into 24 well polystyrene tissue culture trays for compression studies or between two glass plates with a Teflon spacer for tensile studies. All samples were subjected to about 21 hours of freezing at −20° C. and 3 hours of thawing at room temperature for up to ten cycles.

### Determining Mechanical Properties

A bench top mechanical testing machine (Instron Materials Testing System Series 4442 with a 50N load cell) was used to test cylindrical specimens in unconfined compression. Tests were performed in PBS solution at about 37° C. Each sample was compressed to about 30% strain at a strain rate of 100% per minute. The sample size was approximately 15 mm in diameter and 7 mm in height.

Rectangular specimens were tested in tension. Tests were performed in air at room temperature after determining that for the characteristic testing times, temperature and testing media did not have an effect on the results. Each sample was subjected to a strain rate of about 10% per minute to 15% strain. The sample size was approximately 5 mm in width and 1.5 mm in thickness with a gauge length of approximately 20 mm. Both compressive and tensile moduli were calculated from the average slope of the initial linear portion (1% to 5% strain) of the stress versus strain curve.

### Determining Molecular Weight Between Cross-Links

Bray and Merrill modified the original Flory-Rehner equation for molecular weight between cross-links (M_{c}), based on rubber elasticity theory, to account for cross-linking within solution [Bray et al., 1973, J Appl Polym Sci. 17:3779-94]. The Bray and Merrill equation is shown below, where v₂ᵣ and v₂ₛ, are polymer volume fractions in the relaxed state and swollen state, respectively, Mₙ is the number-average molecular weight of polymer chains before cross-linking, vₚ is polymer specific volume, V₁ is solvent molar volume, and χ₁ is the chi parameter (1).

$$\frac{1}{{M}_{c}} = \frac{2}{{M}_{n}} - \frac{\frac{{v}_{p}}{{V}_{1}} [ ln ( 1 - {v}_{2 s} ) + {v}_{2 s} + {χ}_{1} {v}_{2 s}^{2} ]}{{v}_{2 r} [ {( \frac{{v}_{2 s}}{{v}_{2 r}} )}^{\frac{1}{3}} - \frac{1}{2} ( \frac{{v}_{2 s}}{{v}_{2 r}} ) ]} \qquad ( 1 )$$

Previous work performed by Bray and Merrill on M_{c} in PVA hydrogels utilized vₚ as 0.788 cc/g, χ₁ as 0.494, and V as 18 cm³/mol [Bray et al., 1973, J Appl Polym Sci. 17:3779-94]. In order to ensure the accuracy of these constants, care was taken to replicate the experimental conditions performed by Bray and Merrill as much as possible by performing equilibrium swelling of PVA hydrogels in water at 30° C. Volume (V) calculations were performed using equation 2:

$$V = \frac{{M}_{a} - {M}_{h}}{{ρ}_{h}} \qquad ( 2 )$$

where Mₐ and Mₕ refer to sample mass in air and in heptane, respectively, and ρₕ is density of heptane, 0.693 g/cm³. Volume calculations were performed on the hydrogel in the relaxed state, the swollen state, and the dried state in order to determine v₂ᵣ and v₂ₛ. This method was performed for hydrogels at both 10, 20, 30, and 35 wt % PVA, and inbetween one and ten freeze-thaw cycles.

### Determining Bound Water

Another method to evaluate the degree of physical cross-linking within PVA hydrogels is through a measurement of the amount of water bound to the PVA chains. Water is capable of hydrogen bonding to hydroxyl groups on PVA chains. The bound water does not freeze or thaw at the same temperature as unbound water. As hydrogen bonds are formed between the PVA chains, fewer bonding sites are possible for water. Differential scanning calorimetry (DSC) can be used to measure the amount of bound water by analyzing the heat absorbed during melting of unbound water crystals, ΔHₛ as shown in equation 3.

$$Fraction Bound Water = 1 - \frac{Δ {H}_{s}}{Δ {H}_{w}} \qquad ( 3 )$$

The heat of fusion enthalpy of bulk water, ΔH_{w}, was found to be 331 J/g as determined by measuring the melting endotherm of a pure water sample, compared to literature reports of 333.5 J/g [Thomas et al., 2003, J Biomed Mater Res A. 67(4):1329-37]. The amount of bound water was measured for hydrogels between zero and six freeze-thaw cycles for both 10 wt % and 20 wt % PVA hydrogels. Experiments were performed on a TA Instruments Q2000 DSC with a ramp speed of 10° C./min.

### Determining Microstructure

Cyrostat sections, 20 μm thick, were sectioned from cylindrical, hydrated, hydrogel specimens in the direction substantially perpendicular to the cylindrical axis. Optical micrographs using a magnification of 20× were used to analyze hydrogel microstructure inbetween one and six freeze-thaw cycles.

### Fiber-Reinforced PVA Composite Preparation and Evaluation

Melt blown polypropylene mats made of ExxonMobil PP374G homopolymer with a melt flow index of 1500 were used to reinforce the hydrogel. The fiber mats were obtained from Hills, Inc. and had about 1-2 μm diameter fibers. UHMWPE fibers obtained from Fiber Materials, Inc. under trade name Spectra® 1000 in plain weave style 945 with a denier of 215 were also used to reinforce the hydrogel. Composites were prepared using a wet lay-up process by impregnating fiber mats with polymer solution before placing them between two microscope glass slides with a Teflon spacer and securing the assembly with binder clips. Depending on amount of hydrogel solution needed to fill sample volume, additional solution was added as needed. Polymer solutions were prepared as described previously. The mold was subjected to 21 hours of freezing at −20° C. and 3 hours of thawing at room temperature for up to six cycles.

### Determining Tensile Properties

The tensile modulus was measured using rectangular specimens of the composites using a bench top mechanical testing machine. Due to the strength of the UHMWPE fibers, UHMWPE-PVA composites were specially cut using electric shears to avoid damaging the composite. Each sample was subjected to about 15% strain at a strain rate of 10% per minute. PP-PVA composites were tested on an Instron Materials Testing System Series 4442 with a 50N load cell and an approximate sample size of 5 mm in width, 1.5 mm in thickness, and 20 mm in gauge length. UHMWPE-PVA composites had significantly higher moduli then polypropylene composites. As a result, UHMWPE-PVA composites were tested on an Instron Materials Testing System Series 8872 with a 1 kN load cell and an approximate sample size of 12 mm in width, 1.5 mm in thickness, and 30 mm in gauge length. A larger sample size was needed to minimize slipping during testing. Additionally, sand paper on the grip surface was used for both UHMWPE and polypropylene composites to increase effectiveness of the tensile grips. The tensile modulus was calculated from the slope of the linear portion in the stress vs. strain curve from 1 to 5%.

### Determining Microstructure Surrounding UHMWPE Fibers

Cylindrical hydrogel specimens containing a single UHMWPE fiber embedded along the axis of the cylinder were sectioned to 30 μm using a cryostat. The single fiber composite was not capable of being sectioned without debonding the fiber-hydrogel interface. As a result, the fiber was removed slowly from the hydrogel before sectioning to limit PVA deformation near the fiber surface. Sections were performed in a direction perpendicular to the fiber axis. Optical micrographs using a magnification of 20× were used to analyze hydrogel microstructure near the fiber from one to six freeze-thaw cycles.

### Statistical Analysis

All values are reported as mean±standard deviation for at least three independent samples. Statistical analysis was performed using a one-way analysis of variance (ANOVA) followed by a Bonferroni post hoc test with a 95% confidence interval (p<0.05) to compare mean values.

The results of the experiments presented herein are now described.

As described herein, tensile and compression moduli for PVA were evaluated as a function of freeze-thaw cycles and polymer content and compared to the human meniscus. Molecular weight between cross-links and bound water studies were also performed to determine the relationship between degree of cross-linking and hydrogel modulus. Changes in hydrogel microstructure were observed with cycling using optical micrographs and also related to modulus. The range of tensile moduli was analyzed between about 0% and 10% fiber volume for PP-PVA composites, and between about 0% and 29% fiber volume for UHMWPE-PVA composites, to determine the potential of the composites to mimic the anisotropic tensile modulus distribution of the human meniscus. Lastly, the microstructure of the PVA hydrogel surrounding a single UHMWPE fiber was investigated to determine the impact of freeze-thaw cycles on the fiber-gel interface.

### Example #1

### PVA Hydrogel

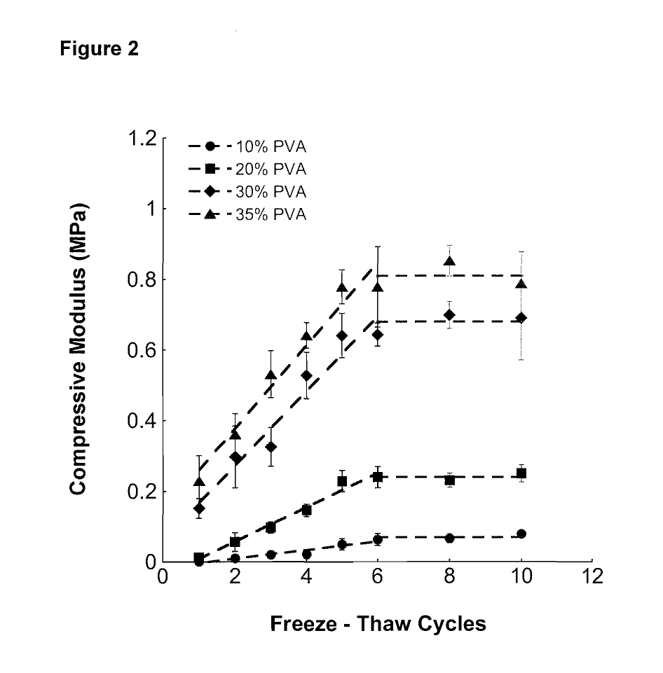

Compressive and tensile testing was performed on PVA hydrogels synthesized from one to ten freeze-thaw cycles. As depicted in FIG. 2, compressive modulus values were calculated as the average slope of the stress-strain curves between 1% and 5% strain. For all PVA concentrations, linear trends in modulus were observed through the first six freeze-thaw cycles. After six cycles, the compressive modulus did not increase significantly (p<0.05), and reached a plateau value. Also depicted in FIG. 2 are trend lines representing the best fit through the first six freeze-thaw cycles and the average modulus within the plateau region. Polymer concentration had a significant effect on compression modulus (p<0.05). Notably, the compressive modulus for 20 wt % PVA is within the range of the native meniscus, which has an aggregate compressive modulus of 0.22 MPa [Almarza et al., 2004, Ann Biomed Eng. 32(1):2-17].

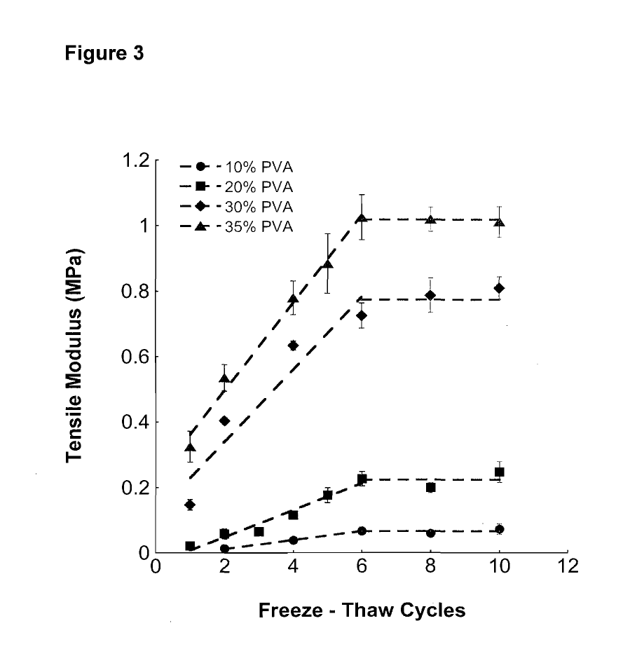

Tensile modulus values were also calculated as the average slope of the stress versus strain curves between about 1% and 5% strain, and are plotted as a function of polymer content and number of cycles in FIG. 3. Trend lines are again shown representing the best fit through the first six freeze-thaw cycles and the average modulus within the plateau region. As expected, poor properties in tension for the PVA hydrogels were observed compared to the range desired for the human meniscus, of about 2 to 295 MPa [Almarza et al., 2004, Ann Biomed Eng. 32(1):2-17]. Tensile modulus behavior exhibited similar trends and values as a function of both polymer content and cycling as those observed in compression, as linear trends were observed through the first six freeze-thaw cycles.

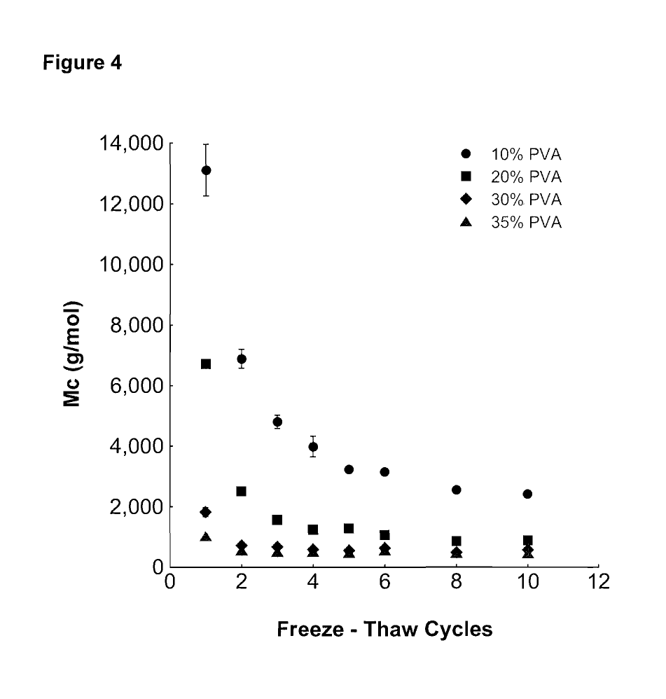

The degree of cross-linking in PVA hydrogels was evaluated using the Bray and Merrill equation, with the results depicted in FIG. 4. Compressive and tensile data, presented herein and confirmed by other work, suggests that cross-linking density increases with freeze-thaw cycles [Hassan et al., 2000, Adv Polym Sci. 153:37-66; Lozinsky et al., 2008, Colloid J.

70(2):189-98; Ricciardi et al., 2004, Macromolecules. 37:9510-6; Hatakeyema et al., 2005, Thermochim Acta. 431; 144-8]. The trend observed between Mc and freeze-thaw cycles supports the presence of an increasing number of cross-links with increasing cycles. Changes in Mc appeared to decrease most significantly in the first few cycles, with no significant change in Mc after four cycles (p<0.05). Additionally, polymer concentrations higher than 20 wt % PVA did not show any significant changes after the first three cycles (p<0.05). When compared to the progressive linear trend observed in both the tensile and compressive moduli through the first six cycles, the increase in cross-linking at these cycles seems unlikely as an explanation of the increases in modulus observed. This suggests the inability of rubber elasticity theory to completely explain the behavior of this system, perhaps as a result of phase separation and the presence of additional mechanisms contributing to the modulus. Furthermore, more cross-linking was observed in samples with higher concentrations of polymer, as expected.

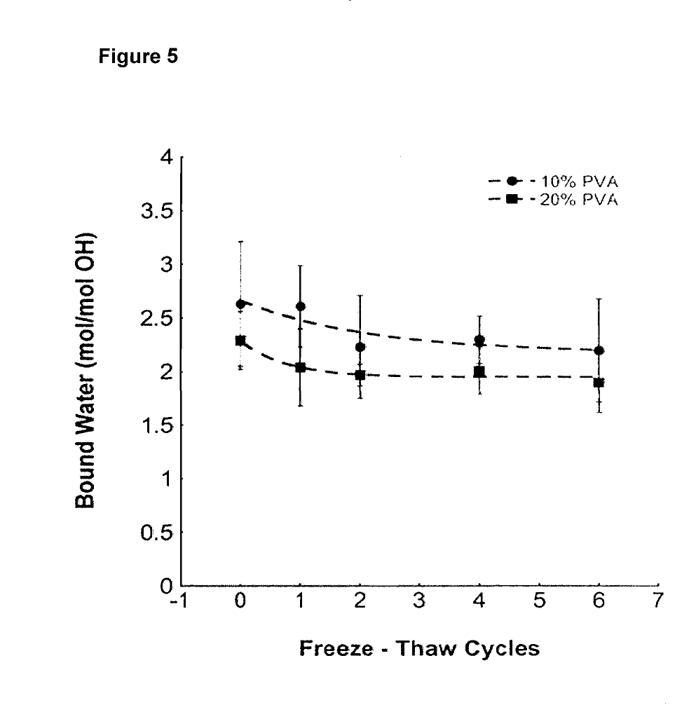

Also demonstrated herein is the determination of the amount of bound water in the hydrogel to support the calculated M_{c} values. Bound water, meaning water that is hydrogen bonded to PVA, does not freeze or thaw at the same temperature as unbound water. Only limited research has been reported on the characterization of bound water within PVA hydrogel networks [Ping et al., 2001, Polymer. 42:8461-7; Cha et al., 1993, Makromol Chem. 194:2433-41]. Furthermore, most of the reported work is for chemically cross-linked PVA systems or systems with a constant number of physical cross-links. As demonstrated herein, the measurement of bound water was made as a function of freeze-thaw cycles to elucidate the effect of freeze-thaw cycles on the development of physical cross-links. The amount of bound water was determined by melting the frozen, unbound, water crystals in the hydrogel. FIG. 5 depicts a plot of bound water measured as mole of water per mole of —OH in PVA hydrogels with freeze-thaw cycling. Bound water shows a similar trend as a function of freeze-thaw cycles and polymer content when compared to M_{c}, as depicted in FIG. 4. That is, decrease in bound water with cycling indicates that hydrogen bonding was occurring between the PVA chains, and reducing the ability of the water to bind to the hydroxyl groups on PVA. Notwithstanding the large, relative standard deviations, decreases in bound water were seen through the first two cycles. The early decreases support the trend observed in M_{c}, but does not correspond to the trends observed in modulus. In fact, modulus continued to increase after 2 cycles.

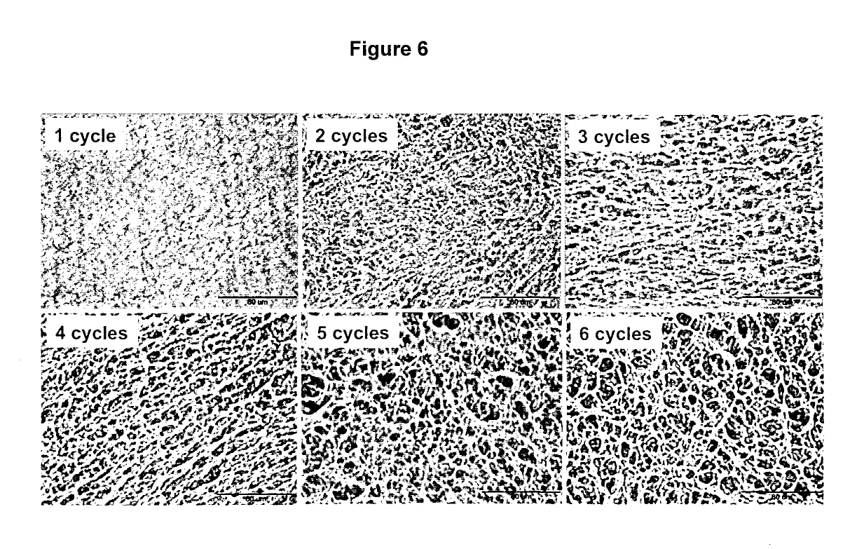

Investigation of the PVA hydrogel microstructure showed increasing pore size and a corresponding thickening of PVA rich regions with freeze-thaw cycles. The representative micrographs of PVA cross-sections as a function of freeze-thaw cycles are depicted in FIG. 6. Microstructure remodeling of the PVA phase occurs as the water freezes, expelling PVA and creating regions of high concentration of polymer. This process repeats with each cycle, creating regions with increasing concentrations of PVA. Additionally, with each cycle, the water freezes and expands, pushing the PVA chains into closer contact. This mechanism is also believed to decrease the distance between the PVA chains, facilitating hydrogen bonding and crystallite formation. The macroscopic remodeling of the polymer rich phase showed a progressive increase of polymer rich zones through the first six cycles, where even after four and five cycles differences in microstructure could be observed. The morphology that developed was a pore structure with pore sizes of approximately 10 μm. The morphology also showed macroscopic phase separation and provides some insight as to why the cross-link data obtained from swelling experiments did not match modulus measurement trends.

### Example 2

### Fiber Reinforced PVA Composite

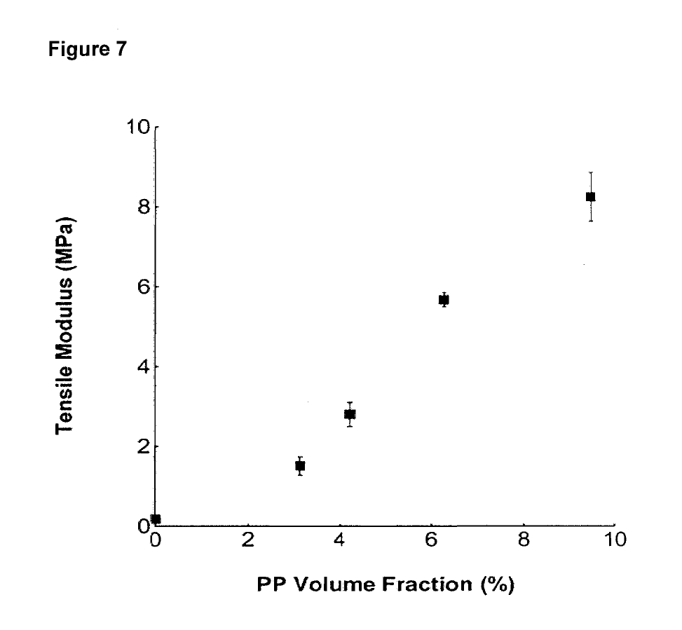

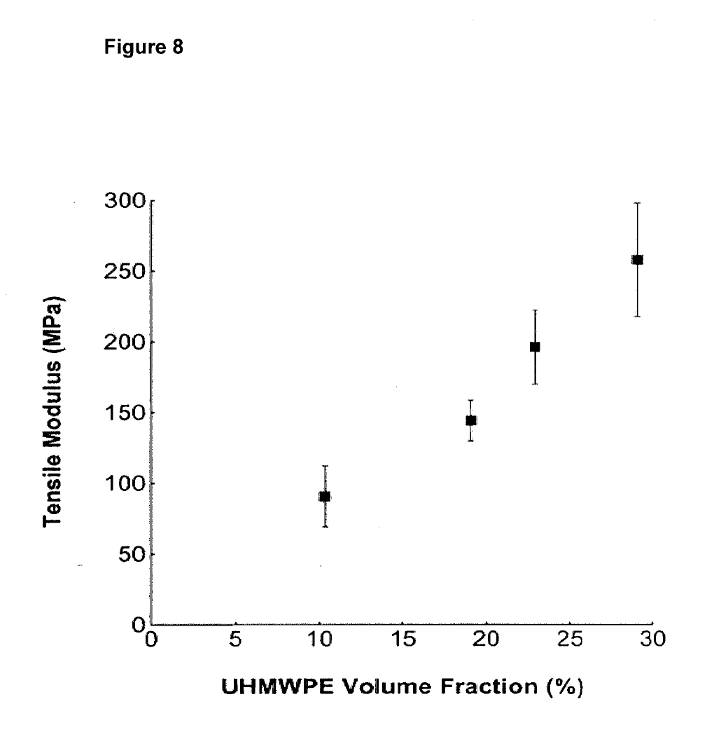

Tensile testing was performed on PVA hydrogels reinforced with polypropylene and UHMWPE fibers. Tensile modulus values were calculated from the average slope of the linear region in the stress versus strain curve. Depicted in FIGS. 7 and 8 are plots of tensile modulus as a function of fiber volume fraction (V_{f}) for polypropylene and UHMWPE fiber-reinforced hydrogels, respectively. Linear increases in modulus as a function of fiber volume fraction were observed in both cases, for the range of V_{f} examined. PP-PVA composites showed an increase in modulus from about 0.23±0.02 MPa for neat PVA to about 8.2±0.6 MPa at 10% V_{f} polypropylene. Reinforcement with UHMWPE fabrics resulted in a tensile modulus of about 90.6±21.6 MPa to 258.1±40.1 MPa at volume fractions of approximately 10% and 29% UHMWPE, respectively. UHMWPE-PVA composites exhibited similar tensile stiffness to that seen in the native meniscus in the circumferential direction, which is about 94 to 295 MPa [Almarza et al., 2004, Ann Biomed Eng. 32(1):2-17]. PP-PVA composites evaluated, on the other hand, more closely matched the range of moduli observed in the human meniscus radially, which is about 2 to 23 MPa [Almarza et al., 2004, Ann Biomed Eng. 32(1):2-17].

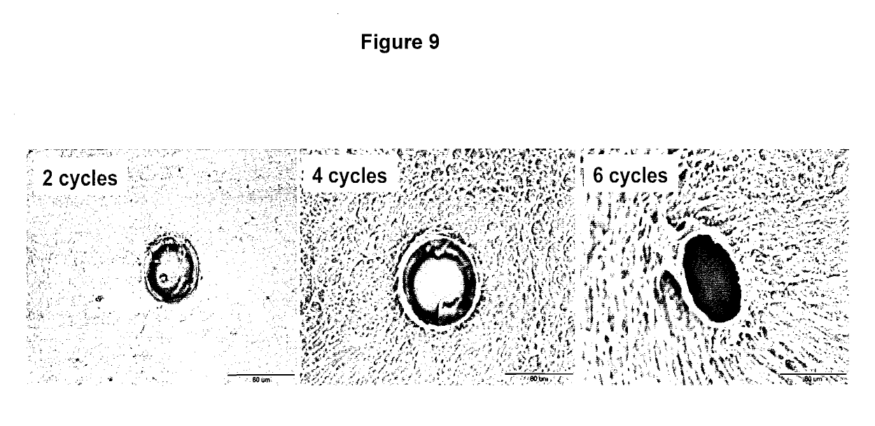

Interfacial stress transfer is an important characteristic of composite materials. In order to gain an understanding of the impact of the structural remodeling occurring within PVA hydrogels during synthesis on the hydrogel-fiber interface, the microstructure of the hydrogels surrounding the UHMWPE fibers was investigated. Cryostat sections substantially perpendicular to a single UHMWPE fiber embedded in a hydrogel were analyzed using optical microscopy, as depicted in FIG. 9. The fiber was pulled out of the hydrogel prior to sectioning to reduce damage to the PVA structure. Interestingly, PVA rich regions appeared to occur around the fiber hole. The fiber hole was significantly larger than the single UHMWPE fibers, which is consistent with poor bonding between the hydrogel and fiber. Fiber hole diameter was not seen to be a function of cycle number, but appears to be completely dependent on initial embedded fiber diameter. This behavior suggests that freeze-thaw cycling increases PVA concentration near the fiber.

As demonstrated herein, the compressive and tensile modulus of PVA hydrogels was evaluated as a function of both freeze-thaw cycles and polymer content. Linear trends for both compressive and tensile modulus were observed with freeze-thaw cycles. Polymer content was also seen to have a significant effect (p<0.05). The formation of physical cross-links during cycling appears to allow the PVA hydrogel network to carry increased load. Physical cross-links are formed between the PVA chains in the form of hydrogen bonding and crystallization. As the water within the sample freezes, regions of very high PVA concentration form, bringing the PVA chains into close contact and allowing for the formation of cross-links. As a result, it is possible to tailor or customize the mechanical properties of a hydrogel through the modification of cycle number, as well as polymer content.

Molecular weight between cross-links and bound water in the hydrogel were determined in order to understand the effect of cross-linking on hydrogel modulus. Both studies reported similar results, in that decreases were observed in the first few cycles with little change after four cycles. In fact, no significant differences were found in M_{c} after three cycles for 20 wt % PVA, and four cycles for 10 wt % PVA. Significant differences, however, were noted through the first six cycles in modulus for both 10 wt % and 20 wt % PVA. This suggests the presence of additional mechanisms contributing to the modulus. Neither swelling nor calorimetric analysis, however, differentiates between physical cross-links formed in amorphous and crystalline regions, and it is possible that the kinetics for the formation of amorphous cross-links and crystalline cross-links are vastly different, and impact the mechanical properties in ways not discernable by these methods. Of importance, rubber elasticity theory used to calculate M_{c} assumes homogenous polymer networks. The inhomogeneous nature of the PVA hydrogel network observed and depicted in FIG. 6 presents questions regarding the applicability of the Bray and Merrill equation to describe cross-links in freeze-thawed PVA hydrogel networks. However, both bound water and M_{c} show similar trends, where bound water was calculated through direct measurement. It should be appreciated that the values reported herein for M_{c} are based on a homogenous network.

Structural remodeling of PVA domains was observed with freeze-thaw cycles. During remodeling, regions of high concentration of PVA formed. The possible formation of highly concentrated regions of amorphous, uncross-linked PVA chains with cycling is a potential factor contributing to the trends observed in the compressive and tensile modulus. It is possible that regions of highly concentrated PVA increase the load bearing ability of the hydrogels, independently of cross-linking.

Fibrous reinforcement of PVA hydrogels was demonstrated using UHMWPE and polypropylene fibers. Results indicate fibrous reinforcement is possible as a method of increasing tensile modulus to match that of the native meniscus, or other targeted tissues. The axial elastic modulus of UHMWPE fibers is high (113 GPa), and composite modulus values calculated, based on simple rule of mixture calculations, where it is assumed only half of the UHMWPE fiber volume contribute to the tensile modulus in the direction of testing, result in values that are much higher than were measured. The equation used for predication is shown below (4), where Eₘ, E_{c}, and E_{f}, are the elastic moduli for the matrix, composite, and fiber, respectively.

E_{c}=(1−V_{f})Eₘ+½V_{f}E_{f} (4)

The predicted modulus from such calculations ranged from about 5.8 GPa to 16.4 GPa for approximately 10% and 29% UHMWPE fiber volume fraction, respectively. This may be due to the poor stress transfer through the matrix phase from fiber to fiber because of the relatively weak hydrogel and poor interfacial bonding.

Microstructure changes were observed surrounding a single UHMWPE fiber with freeze-thaw cycling. The formation of PVA rich regions occurred near the fiber as cycling increased. Additionally, fiber hole diameter was observed to be significantly larger than fiber diameter, which may be due to the poor wetting ability of the UHMWPE fibers, and helps explain the discrepancy between predicted and observed composite tensile moduli.

Thus, the fiber-reinforced PVA hydrogels of the present invention replicate the anisotropic modulus distribution present in the native meniscus. As demonstrated herein, the compression modulus for 20 wt % PVA varied between 0.01±0.0005 MPa after one cycle and 0.24±0.03 MPa at six cycles, compared to the reported aggregate compressive modulus for the human meniscus of 0.22 MPa [Almarza et al., 2004, Ann Biomed Eng. 32(1):2-17]. Fibrous reinforcement of hydrogels increased the tensile modulus from 0.23±0.02 MPa without any reinforcement to 8.2±0.6 MPa and 258.1±40.1 MPa with 10% polypropylene and 29% UHMWPE respectively. The tensile modulus of the human meniscus can range from 2 MPa to 295 MPa [Almarza et al., 2004, Ann Biomed Eng. 32(1):2-17].

### Example #3

### Spectra Fibers Embedded in Physically Cross-Linked PVA

Polymer solutions were prepared by mixing polymer, composed of approximately 99 wt % PVA and 1 wt % poly(vinyl pyrrolidone) (PVP), in deionized water. In other embodiments, it is possible to create polymer solutions with up to about 40 wt % PVA-PVP. PVP was added to the polymer mixture since it has been shown to increase long-term stability (Thomas, et al., 2003, J. Biomed. Mater. Res., 67:1329-1337). The container was sealed and autoclaved at 121° C. for two to six hours to ensure complete dissolution of PVA.

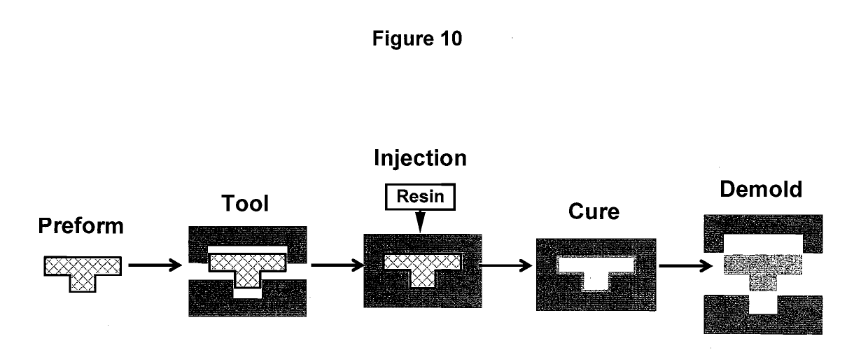

Composites comprised of the hydrogel described above and Spectra fibers obtained in a plain weave were prepared using a resin transfer molding process as outlined in FIG. 10. As contemplated herein, different molds can be used for this process, depending on desired testing. Magnetic resonance imaging can also be used to create custom shaped molds for tissue specific use, and even patient specific use, such as a custom mold for the meniscus of a particular patient. In this embodiment, the resin transfer molding was performed multiple times on samples of increasing size in a multi-stage resin transfer molding process. In the first stage, the fibers were manually placed into the mold and allowed to freeze-thaw for one cycle. In the following stage, the previous stage was used as a guide for fiber placement and allowed to freeze-thaw for one cycle. This process proceeded until the desired size mold was used and allowed to freeze-thaw for the desired number of cycles. A multi-stage process may allow for a more precise placement of fibers throughout the sample during processing.

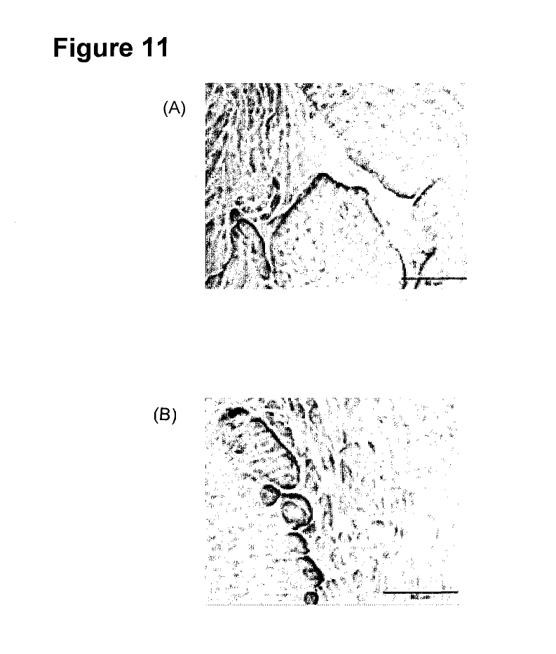

A sample synthesized with two stages without any fibers was analyzed to determine the integrity of the interface between stages. As depicted in FIG. 11, the interface was only visible on a microscopic scale, and in some areas was not visible at all. None of the samples manufactured in this way have shown any failure or weakness at this interface. Spectra fabric was infused with hydrogel resin with a syringe under low pressure.

Samples were subjected to about 21 hours of freezing at about −20° C. and 3 hours of thawing at room temperature for up to ten cycles to induce physical cross-linking. This material was flexible and resistant to tearing.

### Example #4

### Kevlar Fibers Embedded in Chemically Cross-Linked AA-PVA

A precursor solution was prepared by first dissolving poly(vinyl alcohol) (PVA, Elvanol Grade 71-30, Mw˜120,000) in water at about 90° C. for 6 hours. The solution, containing 20% weight PVA, had a viscosity of approximately 100 cp. Acrylic acid was added to the solution to yield about a 1:1 weight ratio of acrylic acid to PVA. Tetraethylene glycol (TEGDMA) was added to the solution as the crosslinking agent in the amount of about 0.75 mol % of the AA monomers. The mixtures were purged with nitrogen to remove any dissolved oxygen, and benzoyl peroxide was added as the reaction initiator in the amount of about 2 wt % of the monomers.

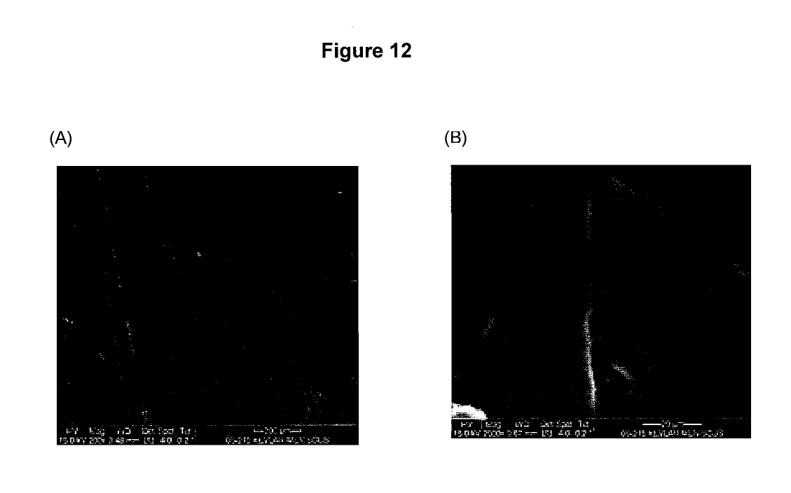

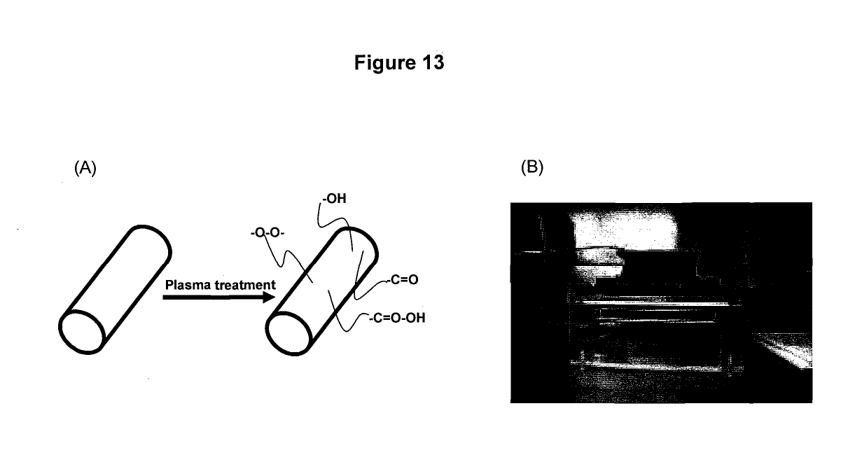

Kevlar composites were created using resin transfer molding. Using SEM, the cross-hatch pattern of fibers embedded within the PVA matrix was seen with fiber diameters of approximately 5-10 microns, as depicted in FIGS. 12a and 12b. The major difference between UHMWPE and Kevlar fibers is chemical structure. Kevlar contains aromatic and amide linkages, while Spectra is comprised of highly oriented aliphatic polyethylene chains containing no heteroatoms. Thus, Kevlar is a more polar material that is easily wet by hydrogels and their monomers, while Spectra fibers require surface modification to impart higher energy species to improve wetting. The surface of Kevlar fibers can be readily modified to include high concentrations of chemically active species using plasma treatment methods. In particular, oxygen plasma introduces a number of oxygen-containing species on the surfaces of aramid fibers, including hydroxyl, carboxylic acid, and peroxy groups. The degree of functionalization depends on the plasma power, time of exposure, and oxygen concentration. Plasma treatments improve interfacial adhesion between Kevlar and thermosetting polymer matrices like epoxies. Plasma surface treatments can also be used to initiate grafting reactions either via direct reaction of the reactive moieties or by activation of the peroxy groups to induce free radical polymerization of vinyl monomers (Brown, et al., 1997, Journal of Materials Science. 32(10):2599-2604; Robinette, et al., 2005, Nuclear Instruments and Methods in Physics Research B, 236:216-222). Furthermore, the surfaces can be modified using plasma treatment, as depicted in FIGS. 13a and 13b, and the surface interactions can be engineered by surface group attachment and polymer grafting. Plasma treatment can also be used to modify the Spectra fibers. Thus, the introduction of the vinyl monomers to treated fibers may provide for covalent attachment of the hydrogel through reaction between reactive groups within the hydrogel and surface modified groups of the fibers. This can lead to the formation of a hydrogel network that is chemically attached to the supporting fibers, and thus preventing fiber-hydrogel delamination.

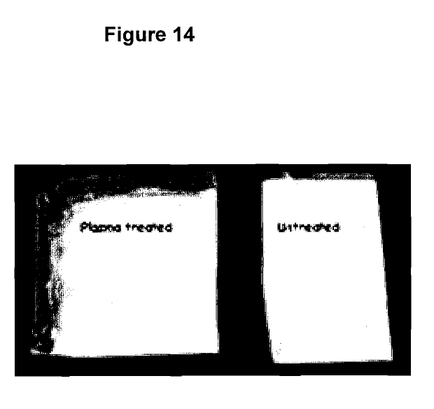

Surface modification of the Spectra fibers was performed prior to the infusion of the hydrogel, via plasma treatment. Plasma treatment was performed at room temperature with a flow rate of 1 LPM O₂ for times up to 9 minutes. Samples manufactured with plasma treatment and without were grossly examined and compared. Air bubbles and composite defects were evident on the untreated samples, but were not apparent on the plasma treated samples, as depicted in FIG. 14.

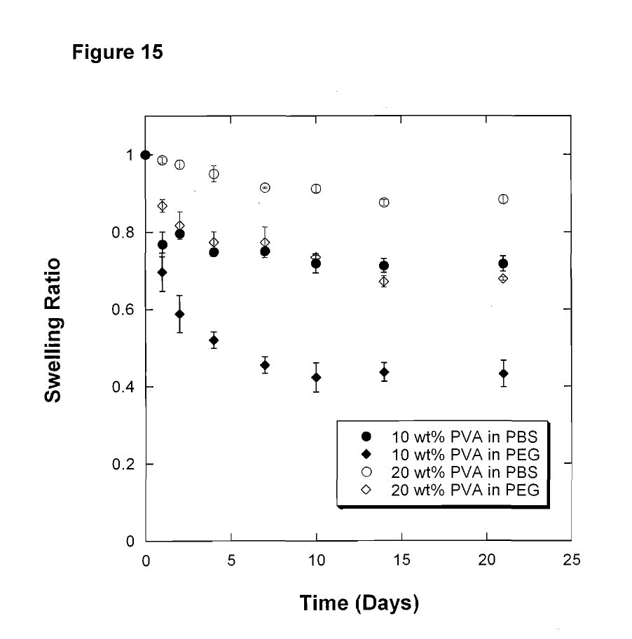

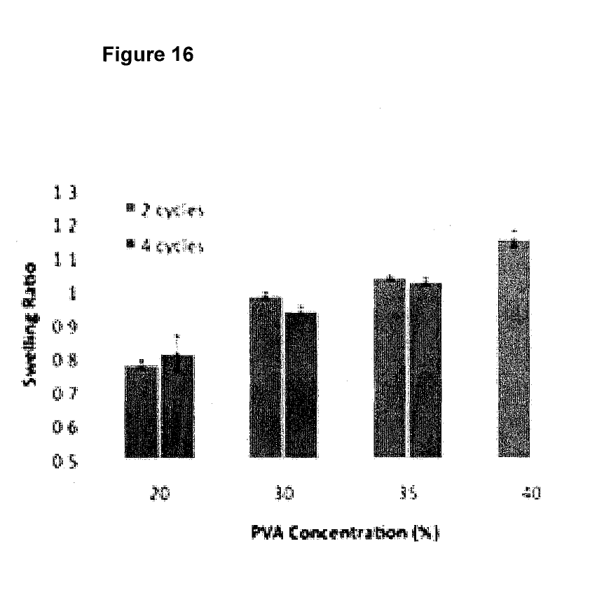

As contemplated herein, the fiber/polymer contents are based primarily on what will be required to prevent in vivo swelling, while maintaining mechanical properties in the range within that of the native tissue, such as the meniscus. As depicted in FIG. 15, in vitro swelling performed in PBS and PEG based solutions show deswelling of PVA hydrogels synthesized using five freeze-thaw cycles. However, samples equilibrated in PEG based solutions all exhibited approximate equilibrium water contents of 32%. This suggests that PVA hydrogels with 32 wt % PVA would exhibit limited volume changes in solution. Preliminary swelling results performed in PEG based solutions agree with this assertion, as depicted in FIG. 16. PEG based solutions with an osmotic pressure of 0.95 atm. were chosen as the best in vitro model due to their ability to mimic the osmotic pressure within the knee (Spiller, et al., 2009, Journal of Biomedical Materials Research. 20B(2):752-759). Approximate fiber volume fraction was determined from preliminary tensile testing results.

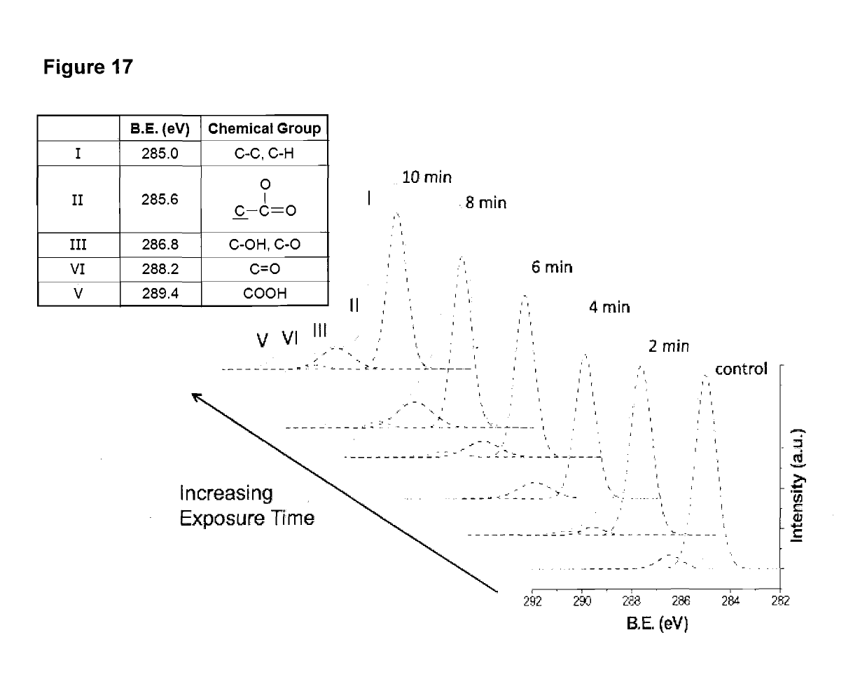

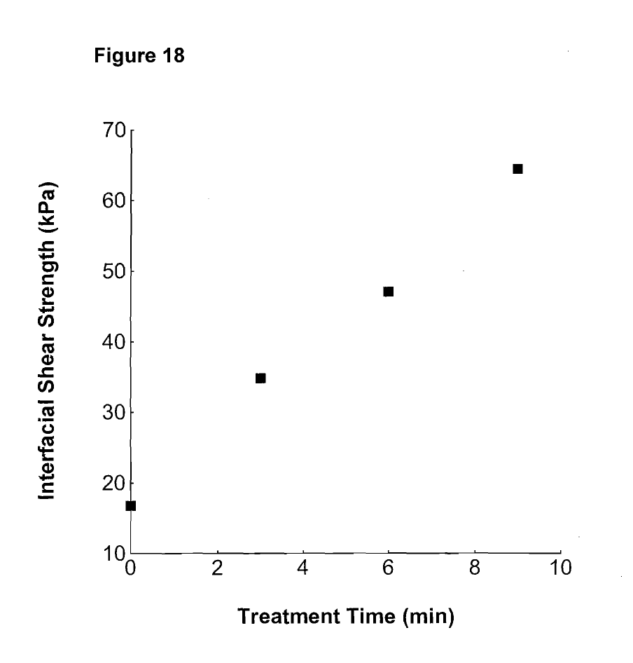

X-ray photoelectron spectroscopy (XPS) results, as depicted in FIG. 17, confirmed the formation of reactive groups on the fiber surfaces following plasma treatment. Fiber pull-out tests showed an increase in interfacial shear strength from 17 kPa to 65 kPa utilizing oxygen plasma treatments, as depicted in FIG. 18.

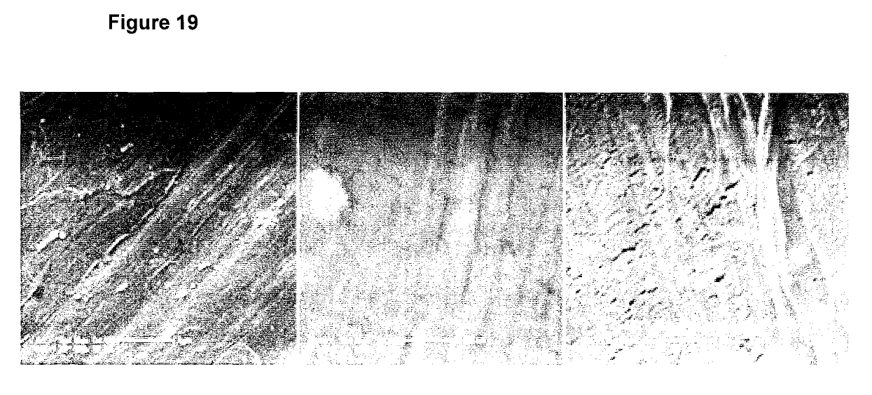

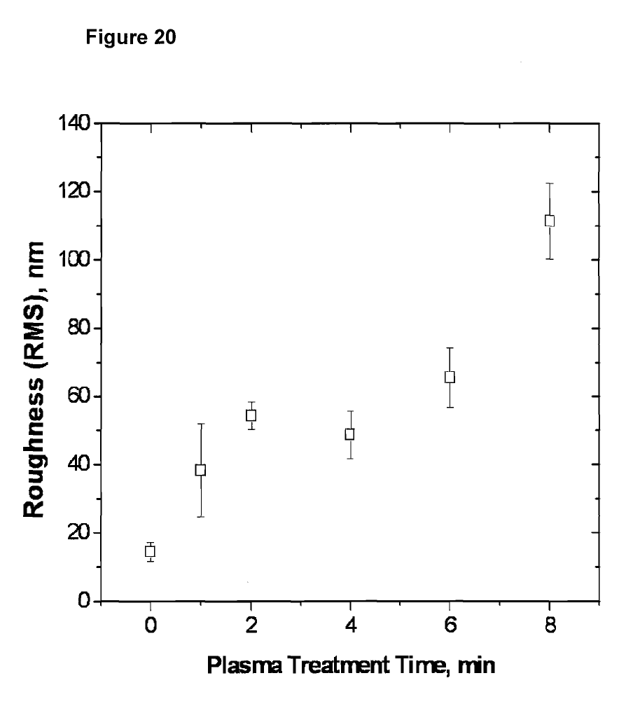

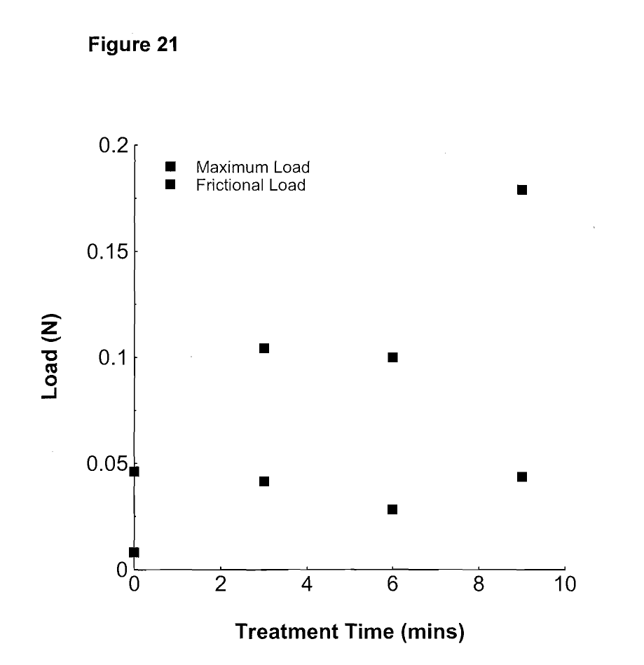

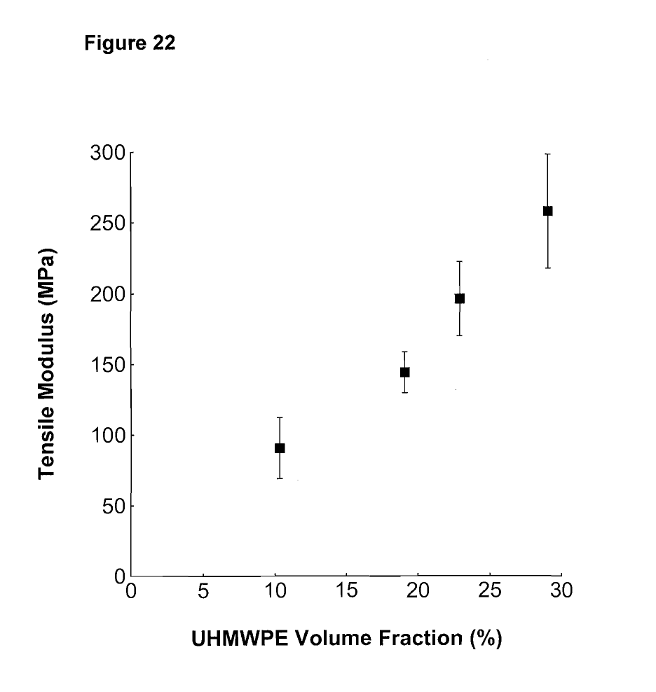

The roughening of UHMWPE fibers was observed using scanning electron microscopy (SEM) (FIG. 19) and atomic force microscopy (AFM) (FIG. 20) and increased with increasing plasma treatment time. Additionally, roughness is believed to have contributed to a 4-fold increase in pull out frictional force found during fiber pull-out testing, as depicted in FIG. 21. A modulus range of 0.25 MPa to 250 MPa can be achieved by changing composite volume fraction from 0% to 28%, as depicted in FIG. 22.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A fiber-reinforced hydrogel composite that mimics a native tissue of a mammal and is suitable for implantation in the mammal, comprising:
at least one fibrous component forming part of a fiber volume fraction;
a polymer fraction comprising poly(vinyl alcohol) (PVA) and poly(acrylic acid) (PAA);
wherein the ratio of PVA to PAA is altered to control the mechanical properties of the composite.

2. The composite of claim 1, wherein mechanical properties of the composite are controlled by altering the amount of cross-linking agent used in cross-linking components of the composite.

3. The composite of claim 1, wherein the at least one fibrous component comprises an ultra high molecular weight polyethylene (UHMWPE).

4. The composite of claim 1, wherein the at least one fibrous component comprises polypropylene (PP).

5. The composite of claim 1, wherein the at least one fibrous component comprises UHMWPE and PP.

6. The composite of claim 1, wherein the fiber volume fraction in the composite is within about 5-50%, and the polymer fraction in the composite is within about 20-50 wt %.

7. The composite of claim 1, wherein at least one of the diameter, orientation, number and volume fraction of the at least one fibrous component is altered to impart directional, anisotropic and inhomogeneous modulus to the composite to produce a tensile modulus range of about 0.25 MPa to 250 MPa, wherein the fiber volume fraction ranges from between about 0% to 28%.

8. The composite of claim 1, wherein the composite osmolality is balanced via the polymer fraction to equilibrate with the mimicked native tissue.

9. The composite of claim 1, wherein the at least one fibrous component is used for attachment upon implantation within the mammal.

10. The composite of claim 1, wherein the composite replaces a musculoskeletal tissue of the mammal.

11. The composite of claim 1, wherein the composite augments a damaged or degraded musculoskeletal tissue of the mammal.

12. The composite of claim 1, wherein the composite replaces the meniscus of the mammal.

13. The composite of claim 1, wherein the composite replaces a ligament or a tendon of the mammal.

14. The composite of claim 1, wherein the composite is used for closure of the annulus pulposus.

15. The composite of claim 1, wherein the composite further comprises a porous periphery to augment attachment within the mammal via an ingrowth of surrounding tissue.

16. A device for implantation within a subject, comprising:
at least one fibrous component forming part of a fiber volume fraction;
at least one hydrogel component forming part of a polymer fraction;
wherein the device is subjected to a plurality of freeze-thaw cycles, such that the mechanical properties of the device are controlled based upon the number of freeze-thaw cycles to which the device is subjected.

17. The device of claim 16, wherein the mechanical properties are further controlled based upon the duration and rate of freezing and thawing during the freeze-thaw cycles.

18. The device of claim 16, wherein the at least one fibrous component comprises an ultra high molecular weight polyethylene (UHMWPE).

19. The device of claim 16, wherein the at least one fibrous component comprises polypropylene (PP).

20. The device of claim 16, wherein the fiber volume fraction in the device is within about 5-50%, and the polymer fraction in the device is within about 20-50 wt %.

21. The device of claim 16, wherein at least one of the diameter, orientation, number and volume fraction of the at least one fibrous component is altered to impart directional, anisotropic and inhomogeneous modulus to the device to produce a tensile modulus range of about 0.25 MPa to 250 MPa, wherein the fiber volume fraction ranges from between about 0% to 28%.

22. The device of claim 16, wherein the device osmolality is balanced via the polymer fraction to equilibrate with a mimicked native tissue of the subject.

23. The device of claim 16, wherein the at least one fibrous component is used for attachment upon implantation within the subject.

24. The device of claim 16, wherein the device replaces a musculoskeletal tissue of the subject.

25. The device of claim 16, wherein the device augments a damaged or degraded musculoskeletal tissue of the subject.

26. The device of claim 16, wherein the device replaces the meniscus of the subject.

27. The device of claim 16, wherein the device replaces a ligament or a tendon of the subject.

28. The device of claim 16, wherein the device is used for closure of the annulus pulposus.

29. The device of claim 16, wherein the device further comprises a porous periphery to augment attachment within the subject via an ingrowth of surrounding tissue.

30. A fiber-reinforced hydrogel composite that mimics a native tissue of a mammal and is suitable for implantation in the mammal, comprising:
at least one fibrous component forming part of a fiber volume fraction;
at least one hydrogel component forming part of a polymer fraction;
wherein the mechanical properties of the composite are controlled based on the selection of the at least one hydrogel component.

31. The composite of claim 30, wherein the selection of the at least one hydrogel component is based on hydrogel viscosity.

32. The composite of claim 30, wherein the selection of the at least one hydrogel component is based on hydrogel stability, such that the hydrogel does not substantially swell or shrink when implanted within the mammal.

33. The composite of claim 30, wherein the selection of the at least one hydrogel component is based on its reaction with the surface of the at least one fibrous component.

34. The composite of claim 30, wherein the mechanical properties of the composite are further controlled based on the selection of the at least one fibrous component.

35. The composite of claim 34, wherein the selection of the at least one fibrous component is based on the ability to weave the at least one fibrous component in a controlled fiber orientation.

36. The composite of claim 34, wherein the selection of the at least one fibrous component is based on the ability to modify the surface of the at least one fibrous component, such that it interacts with the at least one hydrogel component.

37. The composite of claim 34, wherein the at least one fibrous component comprises an ultra high molecular weight polyethylene (UHMWPE).

38. The composite of claim 34, wherein the at least one fibrous component comprises polypropylene (PP).

39. The composite of claim 30, wherein the at least one hydrogel component comprises poly(vinyl alcohol) (PVA) and poly(acrylic acid) (PAA), and wherein the mechanical properties of the composite are further controlled based on the ratio of PVA to PAA.

40. A fiber-reinforced hydrogel composite that mimics a native tissue of a mammal and is suitable for implantation in the mammal, comprising:
at least one fibrous component forming part of a fiber volume fraction;
at least one hydrogel component forming part of a polymer fraction;
wherein the hydrophobic and hydrophilic interactions at the interface of the at least one fibrous component and at least one hydrogel component are maximized; and
wherein in vivo swelling of the composite is minimized.

41. The composite of claim 40, wherein the at least one fibrous component comprises an ultra high molecular weight polyethylene (UHMWPE).

42. The composite of claim 40, wherein the at least one fibrous component comprises polypropylene (PP).

43. The composite of claim 40, wherein the at least one hydrogel component is poly(vinyl alcohol) (PVA) and poly(acrylic acid) (PAA).

44. The composite of claim 43, wherein the mechanical properties of the composite are altered based on the ratio of PVA to PAA.

45. The composite of claim 40, wherein the fiber volume fraction in the composite is within about 5-50%, and the polymer fraction in the composite is within about 20-50 wt %.

46. A method of controlling the mechanical properties of a fiber-reinforced hydrogel composite that mimics a native tissue of a mammal and is suitable for implantation in the mammal, comprising selecting at least one fibrous component in a fiber volume fraction and at least one hydrogel component in a polymer fraction, such that the hydrophobic and hydrophilic interactions at the interface of the at least one fibrous component and at least one hydrogel component are maximized and in vivo swelling of the composite is minimized.

47. The method of claim 46, wherein the fiber volume fraction in the composite is within about 5-50%, and the polymer fraction in the hydrogel is within 20-50 wt %

48. The method of claim 44, wherein the interface is enhanced with a cross-reaction between reactive groups in a precursor mixture and on the fiber surfaces, and wherein the cross-reaction is initiated with a plasma treatment having a duration between about 30 seconds and 10 minutes, and having a plasma flow rate between about 1 and 10 liters per minute oxygen gas.

49. The method of claim 48, wherein the cross-reaction is enhanced with chemical grafting following oxygen plasma treatment.

50. The method of claim 46, wherein the interface is formed via physical interactions between the hydrogel side groups and the fiber surface, such that the physical interaction takes the form of roughened fiber surfaces.

51. The method of claim 46, wherein at least one of the diameter, orientation, number and volume fraction of the at least one fiber component is altered to impart directional, anisotropic and inhomogeneous modulus to the composite to produce a tensile modulus range of about 0.25 MPa to 250 MPa, wherein the fiber volume fraction ranges from between about 0% to 28%.

52. The method of claim 46, wherein the composite osmolality is balanced via the at least one hydrogel component to equilibrate with the mimicked native tissue.

53. The method of claim 46, wherein the at least one fibrous component is used for attachment upon implantation within the mammal.

54. The method of claim 46, wherein the composite replaces a musculoskeletal tissue.

55. The method of claim 46, wherein the composite augments a damaged or degraded musculoskeletal tissue.

56. The method of claim 46, wherein the composite replaces the meniscus.

57. The method of claim 46, wherein the composite replaces a ligament or a tendon.

58. The method of claim 46, wherein the composite is used for closure of the annulus pulposus.

59. The method of claim 46, wherein the composite further comprises a porous periphery to augment attachment within the mammal via an ingrowth of surrounding tissue.

60. The method of claim 46, wherein magnetic resonance imaging scans are used to create custom molds for generating subject-specific implants.

61. The method of claim 46, wherein the composite is manufactured using a multi-stage process.

62. The method of claim 46, wherein the operative technique used to prepare the site for implantation within the mammal is based on computer navigation or computer guided technology.

63. A method of controlling the mechanical properties of a fiber-reinforced hydrogel composite that mimics a native tissue of a mammal and is suitable for implantation in the mammal, comprising:
adding at least one fibrous component in a fiber volume fraction;
adding a plurality of hydrogel components in a polymer fraction, wherein the plurality of hydrogel components comprises poly(vinyl alcohol) (PVA) and poly(acrylic acid) (PAA); and
altering the ratio of PVA to PAA to control the mechanical properties of the composite.

64. The method of claim 63, wherein the mechanical properties of the composite are further controlled by altering the amount of cross-linking agent used in cross-linking components of the composite.

65. The method of claim 63, wherein the mechanical properties of the composite are further controlled by altering at least one of the diameter, orientation, number and volume fraction of the at least one fibrous component to impart directional, anisotropic and inhomogeneous modulus to the composite to produce a tensile modulus range of about 0.25 MPa to 250 MPa, wherein the fiber volume fraction ranges from between about 0% to 28%.

66. A method of controlling the mechanical properties of a fiber-reinforced hydrogel composite that mimics a native tissue of a mammal and is suitable for implantation in the mammal, comprising:
adding at least one fibrous component in a fiber volume fraction;
adding at least one hydrogel component in a polymer fraction; and
subjecting the composite to a plurality of freeze-thaw cycles, such that the mechanical properties of the composite are controlled based upon the number of freeze-thaw cycles the composite is subjected to.

67. The method of claim 66, wherein the mechanical properties are further controlled based upon the duration and rate of freezing and thawing during the freeze-thaw cycles.

68. The method of claim 66, wherein the at least one hydrogel component comprises poly(vinyl alcohol) (PVA) and poly(acrylic acid) (PAA).

69. The method of claim 68, further comprising altering the ratio of PVA to PAA to further control the mechanical properties of the composite.
